(19) United States
(12) Plant Patent Application Publication
(10) Pub. No.: US 2019/0059185 P1
(43) Pub. Date: Feb. 21, 2019

(54) Mango plant named 'NMBP-1243'

(50) ;

(71) Applicant: Commonwealth Scientific and Industrial Research Organization, Acton Act (AU)

(72) Inventor: Ian Stephen Edward Bally, Mareeba (AU)

(73) Assignee: Commonwealth Scientific and Industrial Research Organization, Acton Act (AU)

(21) Appl. No.: 15/731,843

(22) Filed: Aug. 15, 2017

(51) Int. Cl. A01H 6/00

(52) U.S. Cl. PLT/159

(57) ABSTRACT

A new and distinct variety of Mango plant, herein referred to by its cultivar name, ‘NMBP-1243’, is provided which forms fruit with an average weight of 507 grams with skin that is strong red/pink blush over a pale yellow background in coloration. The new variety is an early season variety.

Botanical/commercial classification: Latin name—Mangifera indica L. Common name—Mango Plant. Varietal denomination—‘NMBP-1243’.

### SUMMARY OF THE INVENTION

The new variety of Mangifera indica Mango plant of the present invention was created by closed pollination using hand pollination techniques in 1995 in Darwin, Northern Territory, Australia wherein two parents which previously had been studied were crossed in the hope that they would contribute the desired characteristics. The new cultivar was selected after comparative evaluation of tree and fruit characteristics over several seasons at two sites: Mareeba, Queensland and Darwin, Northern Territory in Australia. The female parent (i.e., seed parent) of the new variety was the ‘Irwin’ variety (non-patented). The male parent (i.e., pollen parent) was the ‘Kensington Pride’ variety (non-patented).

The parentage can be summarized as follows:

‘Irwin’ x ‘Kensington Pride’.

The seeds resulting from the above pollination were sown and small plants were obtained which were physically and biologically different from each other. The new variety was grafted on the ‘Kensington Pride’ variety in early 1997 and field planted at Southedge research station, Queensland, Australia on Jun. 17, 1997. The candidate cultivar was selected after comparative evaluation of tree and fruit characteristics over several seasons. Selective study resulted in the identification of a single plant of the new variety.

It was found that the new Mango plant of the present invention possesses the following combination of characteristics:
(a) displays a medium vigor plant with an open canopy,
(b) exhibits a medium to heavy yielding with a consistent yield year-to-year,
(c) produces monoembryonic seed,
(d) has a harvest season that is early season, and
(e) provides a flavor similar to the ‘Kensington Pride’ variety.

The new variety well meets the needs of the horticultural industry.

The new variety can be readily distinguished from its ancestors. More specifically, the ‘Irwin’ variety displays an erect to horizontal main branch and the young leaf has a weak to medium intensity of anthocyanin coloration, and the ‘Kensington Pride’ variety provides an attenuate shape of the fully developed leaf tip and has no lumpiness on ventral shoulder of mature fruit, whereas the new variety displays an intermediate to spreading attitude of the main branch, the young leaf has a medium to strong intensity of anthocyanin coloration, provides an acute leaf apex shape, and has lumpiness on the ventral shoulder of mature fruit. Additionally, the new variety can be readily distinguished from other known cultivars. For example, the ‘B74’ (U.S. Plant Pat. No. 17,770) variety provides low to moderate tree vigor, while the new variety is a medium vigor plant; the ‘Tommy Atkins’ variety (non-patented) displays fruit which matures late in the season, while the new variety has fruit that matures earlier in the season; and the ‘Delta R2E2’ variety (non-patented) produces polyembryonic seeds, while the new variety is monoembryonic. Also, the ‘NMBP-1201’ variety (U.S. Plant patent application Ser. No. 15/731,850) displays leaves with an acute base shape and an attenuate apex shape, whereas the new variety displays leaves with an obtuse base shape and an acute apex shape.

The new variety has been found to undergo asexual propagation in Australia by a number of routes, including by grafting of cuttings. The typical asexual propagation of the new variety has been to initially increase the numbers of trees for advanced trials and evaluation and more recently for supply of on-farm large scale trials of the variety. Propagation has been by Whip and Tongue or cleft grafting of the new variety (scion) on to a seedling rootstock of the variety ‘Kensington Pride’ in the Mareeba district of Queensland, Darwin in the Northern Territory, or Kununurra in Western Australia. Asexual propagation in Australia has shown that the characteristics of the new variety are stable and strictly transmissible by asexual propagation from one generation to another. Accordingly, the new variety undergoes asexual propagation in a true-to-type manner.

The new variety has been named ‘NMBP-1243’.

### BRIEF DESCRIPTION OF THE PHOTOGRAPHS

The accompanying photographs show, as nearly true as it is reasonably possible to make the same in color illustrations of this character, typical specimens of the new variety.

FIG. 1—illustrates specimens of branches with fruit on an eight year-old tree.

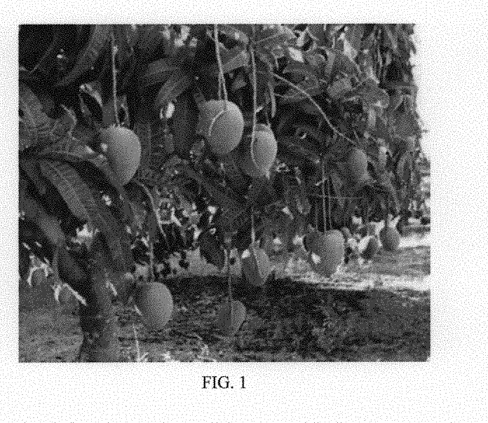

FIG. 2—illustrates a specimen of a mature leaf—upper surface.

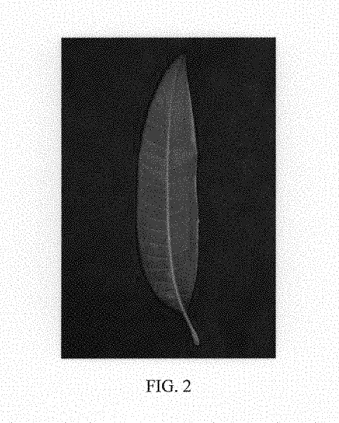

FIG. 3—illustrates a specimen of an immature leaf—lower surface.

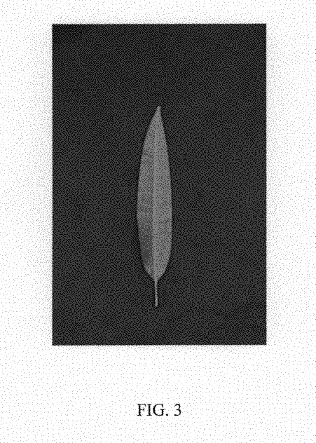

FIG. 4—illustrates a specimen of a single five year-old plant flowering.

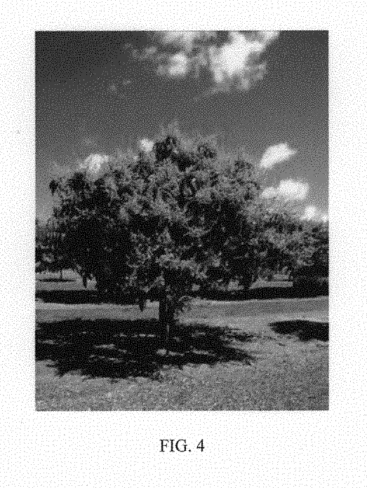

FIG. 5—illustrates a specimen of a single six year-old plant with mature fruit.

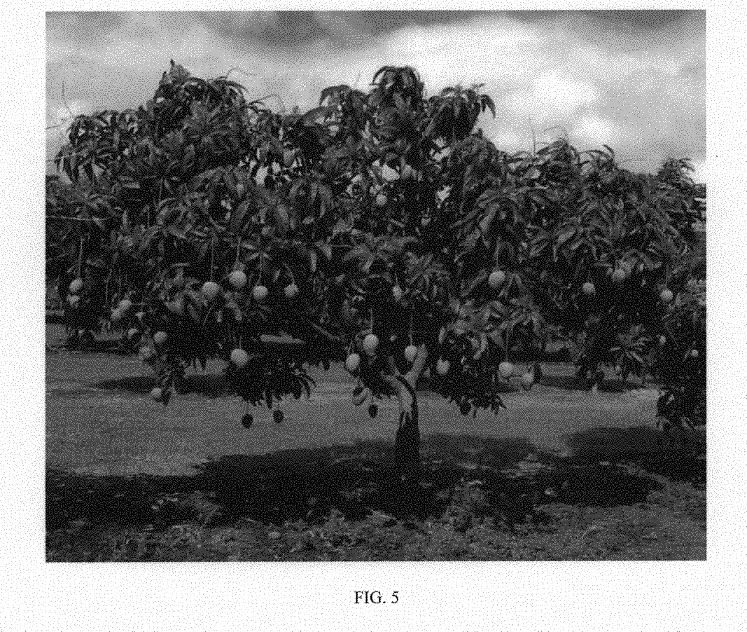

FIG. 6—illustrates specimens of mature, ripening fruit growing on a branch of a six year-old tree.

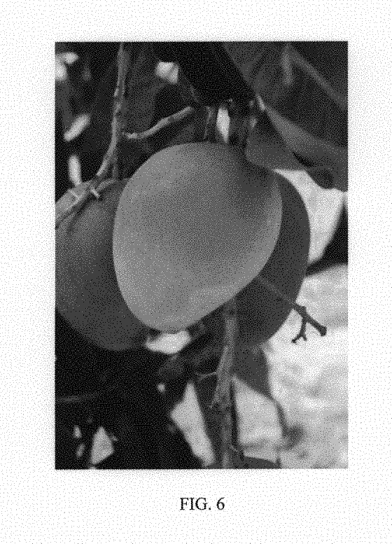

FIG. 7—illustrates a specimen of a ripe fruit, shown as whole and halved and illustrates a specimen of the endocarp and embryo.

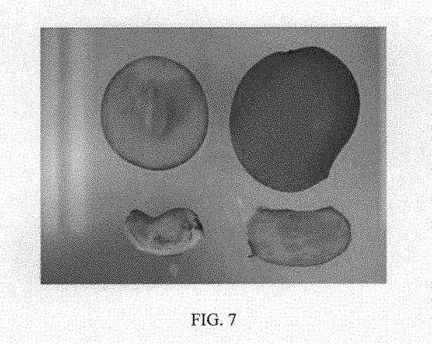

FIG. 8—illustrates a comparison of fruit from plants of the new variety and that of the ‘Kensington Pride’ variety.

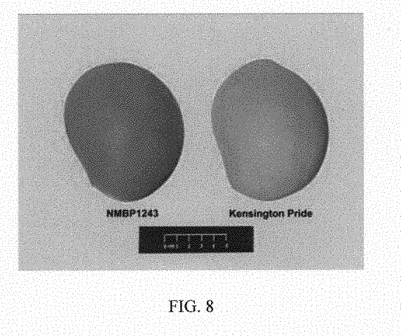

FIG. 9—illustrates specimens of ripe fruit, whole and flesh cut.

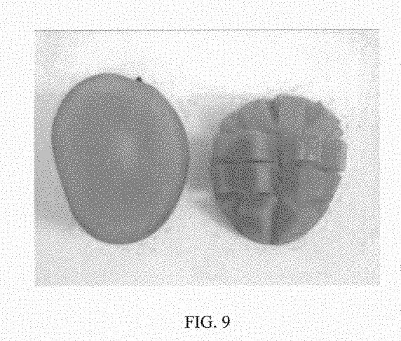

FIG. 10—illustrates specimens of ripe fruit.

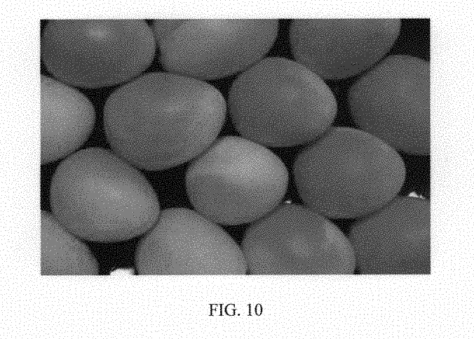

FIG. 11—illustrates a specimen of ripe fruit—side view.

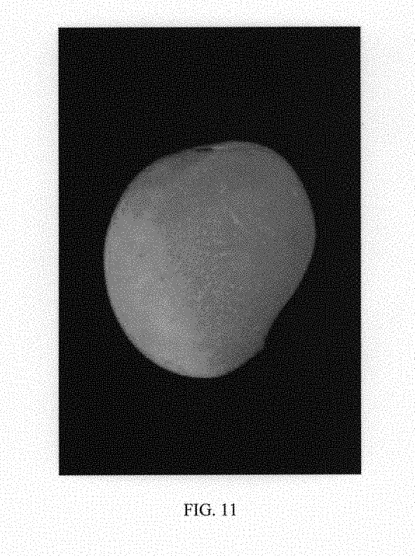

FIG. 12—illustrates a specimen of ripe fruit—halved—displaying flesh and endocarp.

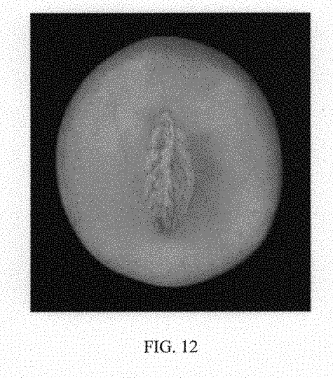

FIG. 13—illustrates a specimen of ripe fruit—top view.

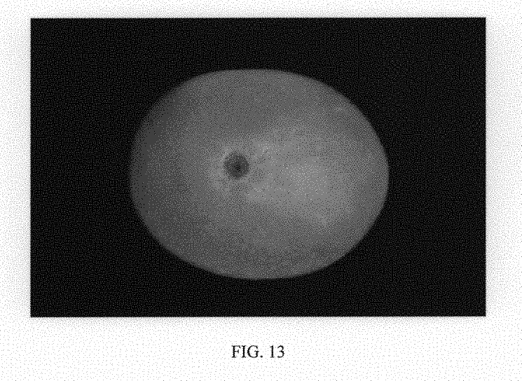

FIG. 14—illustrates a specimen of ripe fruit—front view.

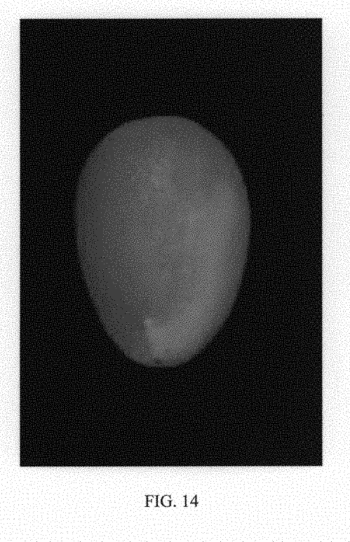

### DETAILED BOTANICAL DESCRIPTION

The chart used in the identification of colors is that of The Royal Horticultural Society (“R.H.S.” Colour Chart), London, England, Fourth Edition 2001. The terminology which precedes reference to the chart has been added to indicate the corresponding color in more common terms. The description is based on a trail design which was a randomized complete block design with five single 8-10 year old tree replicates in Queensland, Australia. 10 to 20 random measurements of each character were assessed on each of the five single tree replicates.

### PLANT

a. Vigor: Commonly medium vigor tree with a medium to open foliage density of the canopy; and the canopy shape is spreading.
b. Attitude of main branches: Horizontal to drooping and intermediate to spreading.
c. Height: Commonly 4 to 6 m in 8 to 10-year-old trees under standard annual pruning.
d. Width: Commonly 4 m in 8 to 10-year-old trees under standard annual pruning.
e. Bark: Commonly no striation and medium rough; texture is smooth; color is commonly near Grey-Brown Group 199B.

### LEAVES

a. Anthocyanin: Present with a medium to strong intensity and reddish coloration on young leaves.
b. Leaf blade:
   Shape.—Generally elliptic with an acute apex and obtuse base, concave in cross section, and is often asymmetric.
   Size.—Length is typically 231.77 mm±12,75 std. dev. on average; width is typically 53.13 mm±2.78 std. dev. on average; and average length to width ratio is typically 4.43:1±0.16 std. dev.
   Relief of upper face.—Raised between secondary veins.
   Undulation of margin.—Weakly present.
   Attitude.—Drooping.
   Color of lower surface of mature leaf.—Commonly near Yellow-Green Group 146B.
   Color of upper surface of mature leaf.—Commonly near Green Group 136B.
   Color of lower surface of immature leaf.—Commonly near Greyed-Red Group 178A.
   Color of upper surface of immature leaf.—Commonly near Greyed-Purple Group 183A.
   Twisting.—Absent.
   Curvature of midrib.—Present and position is commonly from apex.
   Spacing of secondary veins.—Medium.
   Predominant relief of veins on upper surface.—Smooth.
   Serration.—Absent.
   Fragrance.—Present.
   Venation.—Pinnate with medium secondary veins which are approximately 10.14 mm±0.62 std. dev. apart, and the color is commonly near Yellow-Green Group 154C.
c. Petiole:
   Attitude.—Semi-erect.
   Length.—Short, approximately 27.79 mm on average, commonly between 17 and 48 mm.
   Diameter.—Commonly between 2.77 and 3.63 mm.
   Color.—Commonly near Yellow-Green Group N144A.

### INFLORESCENCE

a. Attitude of axis: Horizontal.
b. Size: Length is typically 293.60 mm±16.92 std. dev. on average; width is typically 205.3 mm±12.39 std. dev. on average; and length to width ration is typically 1.54:1±0.14 std. dev. on average.
c. Number of inflorescence branches: 21.91±2.19 std. dev., on average.
d. Color of axis and branches: Commonly near Red-Purple Group 60C.
e. Pubescence on axis and branches: Present in sparse density.
f. Leafy bracts: Present; size, shape and color designation not observed.
g. Flower size: Medium, diameter is approximately 9.26 mm±0.45 std. dev., on average.
h. Number of flowers per inflorescence: Variable commonly between 300 and 1500.
i. Position of fertile stamen(s) in relation to style: Parallel.
j. Length of fertile stamen(s) in relation to style: Equal.
k. Development of staminodes: Very weak to weak.
l. Anthocyanin coloration: Weak to medium intensity present in old flower.
m. Inflorescence type: Narrowly to broadly conical panicle.
n. Pubescence on axis and branches: Commonly sparse.
o. Percentage of hermaphrodite flowers and male flowers pre inflorescence: Varies depending on temperature during inflorescence development.
p. Color of petal pre-anthesis: Commonly near White Group 115A.
q. Color of sepal pre-anthesis: Commonly near Yellow-Green Group 144D.
r. Color of flower peduncle pre-anthesis: Commonly near Yellow-Green Group 144B.
s. Color of wilted petal post-anthesis: Commonly near Red-Purple Group 62B.
t. Color of sepal post-anthesis: Commonly near Yellow-Green Group 144C.
u. Color of flower peduncle post-anthesis: Commonly near Yellow-Green Group 144B.
v. Anthocyanin coloration wilted petal post-anthesis: Present, intensity is weak.
w. Fertile stamen(s): Position in relation to style is parallel, length in relation to style is equal; typically two present.
x. Pistil: Single.
y. Staminodes: Typically 3 present.
z. Filament and style: Color not observed.

### FRUIT

a. Shape: Ovate and deeper than it is wide, with a small sharp beak that flattens out at full maturity, flat or slightly depressed at the stem end.
b. Flavor: Commonly similar to the ‘Kensington Pride’ variety, no turpentine flavor.
c. Mature fruit:
   Size.—Short to medium length, medium width, very low to low length/width ratio, and depth is approximately 85.5 mm on average.
   Weight.—Commonly 319 to 799 g; approximately 507 g on average with approximately 47% 460 to 540 g.
   Shape.—Broad elliptic in cross section ovate and deeper than it is wide, with a small sharp beak that flattens out at full maturity, flat or slightly depressed at the stem end, ventral shoulder is rounded outwards, and dorsal shoulder is rounded downward, with larger fruit rounded upwards.
   Skin color.—Background is commonly near Yellow-Green Group 144A, blush color is commonly near Red-Purple Group 60B over up to 80% of the skin when fully exposed to the sun; only a weak blush is present when fully shaded.
   Bloom on skin.—Conspicuous.
   Stalk cavity.—Present and has a shallow depth.
   Neck.—Absent.
   Groove in ventral shoulder.—Present with a medium length, and medium depth.
   Lumpiness on ventral shoulder.—Present.
   Sinus proximal of stylar scar.—Present with a weak prominence.
   Bulging proximal of stylar scar.—Absent or weak.
   Shape at stylar scar.—Pointed.
   Diameter of stalk.—Medium.
d. Ripe fruit:
   Color of skin.—Predominant orange and red, background is commonly near Yellow-Orange Group 21 B and blush is commonly near Red Group 47A, with a medium degree of speckled pattern of skin coloration.
   Thickness of skin.—Thin to medium; approximately 0.81 mm on average.
   Adherence of skin to flesh.—Medium.
   Main color of flesh.—Commonly near Yellow-Orange Group 21A.
   Firmness.—Approximately 0.81 mm depression, 50 g for 30 sec on average; similar to the “Kensington Pride’ variety; firmness of flesh is soft.
   Juiciness.—Medium.
   Texture of flesh.—Fine to medium.
   Amount of non-fleshy fiber in flesh attached to stone.—Low to medium.
   Amount of fleshy fiber beneath the skin.—Low.
   Pulp.—Color is commonly near Yellow-Orange Group 17A, texture is commonly soft, fiber is very slight, depth is approximately 30 mm on average in a cut cheek.
e. Lenticels: Medium in size with a medium density and commonly light yellow in color, conspicuousness is weak in mature fruit and cause a roughness of surface in mature fruit.
f. Fruit count per panicle: Predominantly single fruit per panicle.
g. Seed:
   Prominence of point at stylar area.—Medium.
   Relief of stone surface.—Smooth.
   Sharp points on surface.—Present on half of the seeds.
   Fibres on cheeks of stone.—Short to medium length and sparse to medium density.
   Texture of fibre.—Medium.
   Endocarp.—Thin, color is not observed.

### DEVELOPMENT

a. Seed embryony: Monoembryonic.
b. Time of fruit maturity: Very early to early, varies with temperature; in Mareeba district of Queensland Australia the typical length of fruit maturing is between two months, two weeks and three months, two weeks.
c. Cropping: Commonly mature grafted trees produce approximately 20-90 kg of fruit per tree each year.
d. Harvest season: Commonly early in the season in Darwin, Kununurra, and Mareeba; typically two to four weeks before the ‘Kensington Pride’ variety.
e. Yield: Commonly medium to heavy, consistent year-to-year.
f. Hardiness: Coldest observed temperature withstood was 4° C.; hottest observed temperature withstood was 39° C.
g. Bloom: Typically between mid-July and late September in Mareeba district of Queensland Australia.
h. Pest/disease resistance: Not observed to date.
i. Shipping quality, fruit storage life, fruit market use: Not observed to date.

Plants of the ‘NMBP-1243’ variety have not been observed under all possible environmental conditions to date. Accordingly, it is possible that the phenotypic expression may vary somewhat with changes in light intensity and duration, cultural practices, and other environmental conditions.

I claim:

1. A new and distinct Mango plant characterized by the following combination of characteristics:
(a) displays a medium vigor plant with an open canopy,
(b) exhibits a medium to heavy yielding with a consistent yield year-to-year,
(c) produces monoembryonic seed,
(d) has a harvest season that is early season, and
(e) provides a flavor similar to the ‘Kensington Pride’ variety;
substantially as herein shown and described.
